(19) United States
(12) Patent Application Publication
(10) Pub. No.: US 2015/0164376 A1
(43) Pub. Date: Jun. 18, 2015

(54) SYSTEM AND DEVICE FOR HEALTHCARE MONITORING

(71) Applicant: International Mobile IOT Corp, Taipei (TW)

(72) Inventor: JUNG-TANG HUANG, Taipei (TW)

(21) Appl. No.: 14/561,229

(22) Filed: Dec. 5, 2014

(30) Foreign Application Priority Data
Dec. 17, 2013 (TW) 102146723

(51) Int. Cl. A61B 5/11; A61B 5/07; A61B 5/00

(52) U.S. Cl. CPC A61B 5/1112; A61B 5/7282; A61B 5/747; A61B 5/0077; A61B 5/1128; A61B 5/6801; A61B 5/076

(57) ABSTRACT

A healthcare monitoring system comprises a server, a node device, a wearable device, and an object device. When the wearable device senses a physiological signal, it generates a physiological parameter based on the physiological signal and sends the physiological parameter to the node device. When the object device senses a non-physiological signal, it generates non-physiological information based on the non-physiological signal, and sends the non-physiological information to the node device. Then, the node device receives and sends the physiological parameter and the non-physiological information to the server, so the server determines health information of a user based on the physiological parameter and the non-physiological information.

### FIELD OF THE INVENTION

The present invention relates generally to a device and a system for monitoring, more specifically a healthcare monitoring system with a wearable device to provide monitoring of location, habit, emotion, and health information of a user of the wearable device.

### BACKGROUND OF THE INVENTION

With the changing social patterns, elder solitary population continues to increase, thus making common disease patterns gradually changed from “acute” to “chronic”, resulting in healthcare costs continue to rise imperceptibly. In order to make more efficient use of medical resources, much more attention has been paid on the approach of home/remote healthcare management.

Traditionally, home/remote healthcare is realized by integration of terminals and non-wearable devices, user activities and habit are not monitored. In this case, changing of user habit or medical compliance makes preventive healthcare difficult to be effective.

From another point of view, rising of medical costs making most people not willing to accept medical examination or treatment when physical discomfort is not felt. Therefore people get disease or even let the disease become worse without knowing they are sick. When symptoms become obvious, medical time and costs required would be increased dramatically. Therefore, an effective healthcare monitoring system for medical resource management and disease prevention is needed to avoid waste of medical resources.

### BRIEF SUMMARY OF THE INVENTION

The present invention provides a healthcare monitoring system that comprises a server, a node device, a wearable device, and an object device. The server comprises a communication module, a processor, and storage connected to the processor and the communication module. The node device comprises a first microcontroller unit (MCU), a first wireless module, and a directional antenna connected to the first MCU and the first wireless module. The wearable device comprises a second MCU, a second wireless module, and a physiological sensor connected to the wearable MCU and the wearable wireless module. The object device comprises an third MCU, a third wireless module, and a non-physiological sensor connected to the third MCU and the third wireless module. When the physiological sensor senses a physiological signal, the second MCU generates a physiological parameter based on the physiological signal, and the second wireless module sends the physiological parameter to the node device. When the non-physiological sensor senses a non-physiological signal, the third MCU generates non-physiological information based on the non-physiological signal, and the third wireless module sends the non-physiological information to the node device. The node device receives and sends the physiological parameter and the non-physiological information to the server by the node device wireless module, wherein the server determines health information of the user by the processor based on the physiological parameter and the non-physiological information.

The present invention also provides a method for healthcare monitoring. The method comprises the following steps: receiving, by a server, a physiological parameter from a wearable device having a physiological sensor; receiving, by the server, a non-physiological information from an object device having a non-physiological sensor; determining, by the server, a health information of a user of the wearable device by a processor based the physiological parameter and the non-physiological information.

The present invention also provides a node device for the healthcare monitoring system. The node device comprises a microcontroller unit (MCU), a wireless module, and a directional antenna connected to the MCU and the wireless module. The directional antenna broadcasts location information to a wearable device, then receives a current location from the wearable device, and sends the current location to a server, wherein the current location is determined by the wearable device according to the location information.

The present invention further provides a wearable device for the healthcare monitoring system. The wearable device comprises a microcontroller unit (MCU), a wireless module, and a physiological sensor connected to the MCU and the wireless module. When the physiological sensor senses a physiological signal, the MCU generates a physiological parameter based on the physiological signal, and the wireless module sends the physiological parameter to a server via a node device, wherein the server determines health information of a user of the wearable device, and sends a notification to the wearable device if the health information is abnormal.

In view of the above, the healthcare monitoring system provides monitoring of a person's location, habit, and health information effectively.

### BRIEF DESCRIPTION OF THE DRAWINGS

The accompanying drawings illustrate one or more embodiments of the invention and together with the written description, serve to explain the principles of the invention. Wherever possible, the same reference numbers are used throughout the drawings to refer to the same or like elements of an embodiment, and wherein:

FIG. 1 is a schematic illustration of the healthcare monitoring system according to one embodiment of the present invention;

FIG. 2 is a schematic illustration of a locating method of the healthcare monitoring system to obtain a current location of a wearable device according to one embodiment of the present invention;

FIG. 3 is a schematic illustration of a healthcare monitoring system according to one embodiment of the present invention;

FIG. 4 is a schematic illustration of the healthcare monitoring system that is installed with one object device at the TV and one object device at the sofa in front of the TV according to one embodiment of the present invention;

FIG. 5 is a schematic illustration of the node device further comprising an environmental sensor and an environmental actuator according to one embodiment of the present invention;

In accordance with common practice, the various described features are not drawn to scale and are drawn to emphasize features relevant to the present disclosure. Like reference characters denote like elements throughout the figures and text.

### DETAILED DESCRIPTION OF THE INVENTION

The present invention will now be described more fully hereinafter with reference to the accompanying drawings, in which exemplary embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like reference numerals refer to like elements throughout.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” or “includes” and/or “including” or “has” and/or “having” when used herein, specify the presence of stated features, regions, integers, steps, operations, elements, and/or components, but do not preclude the presence or addition of one or more other features, regions, integers, steps, operations, elements, components, and/or groups thereof.

It will be understood that the term “and/or” includes any and all combinations of one or more of the associated listed items. It will also be understood that, although the terms first, second, third etc. may be used herein to describe various elements, components, regions, parts and/or sections, these elements, components, regions, parts and/or sections should not be limited by these terms. These terms are only used to distinguish one element, component, region, part or section from another element, component, region, layer or section. Thus, a first element, component, region, part or section discussed below could be termed a second element, component, region, layer or section without departing from the teachings of the present invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

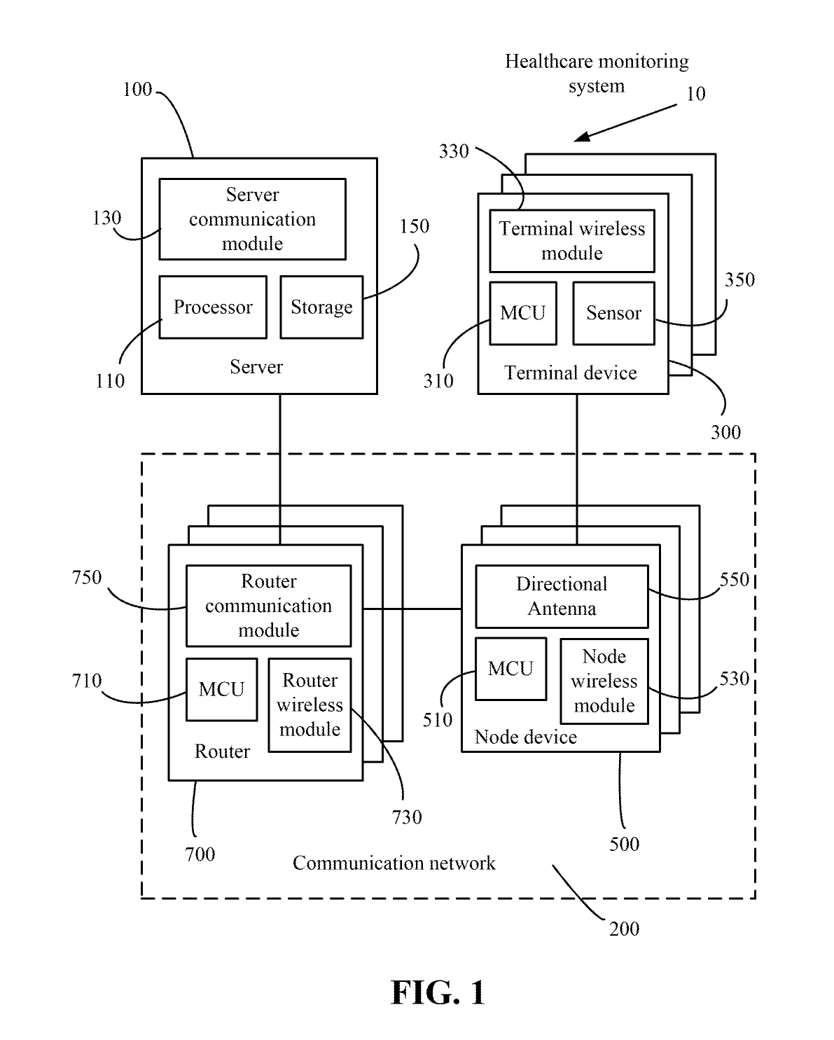

The description will be made as to the embodiments of the present invention in conjunction with the accompanying drawings in FIGS. 1 to 5. Reference will be made to the drawing figures to describe the present invention in detail, wherein depicted elements are not necessarily shown to scale and wherein like or similar elements are designated by same or similar reference numeral through the several views and same or similar terminology.

FIG. 1 schematically shows a healthcare monitoring system 10 according to one embodiment of the present invention. The healthcare monitoring system 10 may comprise a server 100, a communication network 200, and at least one terminal device 300. The at least one terminal device 300 may communicate with the server 100 via the communication network 200, wherein the communication network 200 may comprise at least one node device 500, and at least one router 700. The server 100 may comprise a processor 110, a server communication module 130, and storage 150 connected to one another. The terminal device 300 may comprise a microcontroller unit (MCU) 310, a terminal wireless module 330, and a sensor 350 connected to one another. The node device 500 may comprise a MCU 510, a node wireless module 530, a directional antenna 550 connected to one another. The router 700 may comprise a MCU 710, a router wireless module 730, and a router communication module 750 connected to one another. The terminal device 300 may sense either physiological signal or non-physiological signal with the sensor 350, and the MCU 310 may generate information comprising either physiological parameter or non-physiological information based on the signal sensed by the sensor 350. The terminal wireless module 330 may send the generated information to the at least one node device 500. The at least one node device 500 may receive the generated information by the directional antenna 550, and the MCU 510 may control the node wireless module 530 to send the generated information to the at least one router 700. The at least one router 700 may receive the generated information by the router wireless module 730, and the MCU 710 may control the router communication module 750 to send the generated information to the server 100. The server 100 may receive the generated information by the server communication module 130, and save the generated information to the storage 150 or process the generated information using the processor 110. If the server 100 needs to respond the terminal device 300, transmission would be opposite direction like said above, such as from the server 100 to the router 700, then to the node device 500, and lastly finish at the terminal device 300. In one embodiment of the present invention, the terminal device 300 may send generated information to the server 100 via the at least one node device 500, but without the router 700, wherein the communication network 200 comprises only at least one node device 500. In this case, the server 100 may respond to the terminal device 300 via only the at least one node device 500 without the router 700 as well.

In one embodiment of the present invention, the router 700 may be a gateway (not shown) for accessing the server 100. Alternatively, the router 700 may be a combination of router/gateway (not shown). This approach applies to all embodiment of the present invention.

It should be noted that all communication within the healthcare monitoring system 10 may be wireless or wired except communication from/to the at least one terminal device 300, wherein the communication from/to the at least one terminal device 300 is wireless communication such as Bluetooth, Wi-Fi, Zigbee, RFID, etc. Therefore, the server 100 may communicate with the at least one router 700 by wired or wireless communication, and the same approach applies between the at least one router 700 and the at least one node 500, wherein the at least one node 500 may comprise a node communication module (not shown) for wired communication. Examples of wired communication may be Ethernet, Power Line Communication (PLC), etc.

In one embodiment of the present invention, the healthcare monitoring system 10 may be operated indoor or outdoor. Furthermore, the healthcare monitoring system 10 may be operated partially indoor and partially outdoor. For example, the server 100 may be placed indoor, and the rest of the healthcare monitoring system 10 may be placed outdoor.

In one embodiment of the present invention, the terminal device 300 may be a mobile device, wearable device, implanted device, removable patch device, handheld device, PCB board, or any kinds of module installed on any electrical appliances or furniture, etc. The sensor 350 may be physiological sensor for various physiological parameters of human and/or animal such as body temperature, pulse, respiration rate, blood pressure, blood glucose, blood oxygen, etc. Alternatively, the sensor 350 may be non-physiological sensor such as sensor for proximity, force, pressure, touch, optical, humidity, room temperature, oxygen, carbon dioxide, etc.

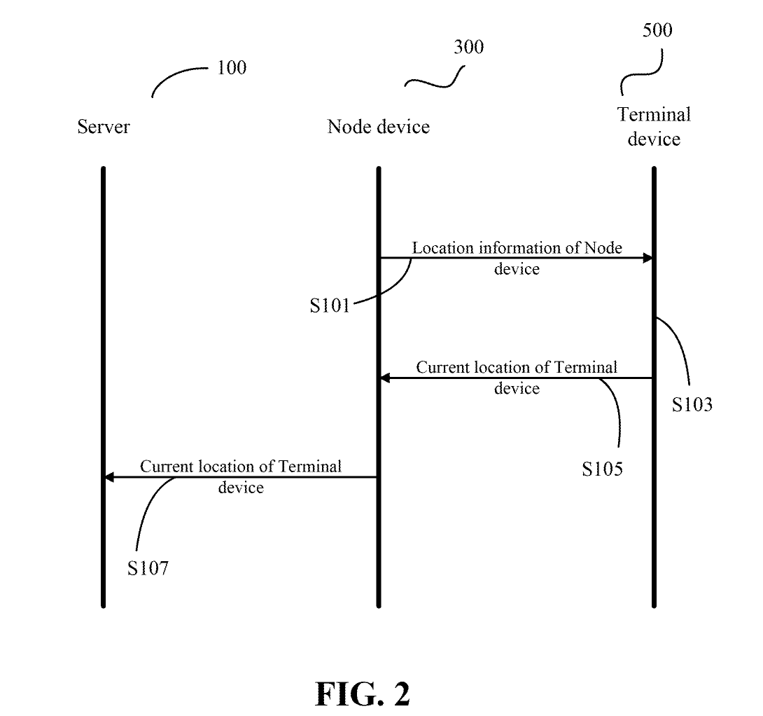

FIG. 2 schematically shows a locating method to monitor location of the terminal device 300 by the healthcare monitoring system 10 according to one embodiment of the present invention. The healthcare monitoring system 10 may monitor the location of the terminal device 300 that is within the communication range of at least one node device 500. The locating method may comprise the following steps:

S101: The at least one node device 500 broadcasts location information to the terminal device 300 by the node wireless module 530, wherein each at least one node device 500 is assigned with a specific location information of its own;

S103: the MCU 310 determines a current location of the terminal device 300 based on the location information and RSSI of the location information;

S105: thereafter the terminal wireless module 330 broadcasts the current location to the at least one node device 500;

S107: the at least one node device 500 sends the current location to the server 100 by the node wireless module 530.

The current location may be determined by trilateration, triangulation, or any other traditional positioning method. Alternatively, a plurality of the node devices 500 may be provided, and each of the directional antenna 550 of each node device 500 may be configured to broadcast the location information without overlapping. For example, the directional antenna 550 may be configured to broadcast downwardly from ceiling to ground in a limited manner such as only terminal device 300 that is below the directional antenna 550 may receive the location information. Therefore, in step 103, the current location may be regarded as the location information.

In one embodiment of the present invention, the RSSI may be replaced by channel response of the transmission between the at least one node device 500 and the terminal device 300.

Although the locating method presented in FIG. 2 does not involve the router 700, it should be kept in mind that involving the router 700 is applicable as such that in the step S107 of the locating method the at least one node device 500 sends the current location to the at least one router 700 by the node wireless module 530, and the at least one router 700 sends the current location to the server 100 by the router communication module 750. Involving the router 700 does not affect the result of practicing the locating method. However, the router 700 may provide more flexibility in selection of communication protocol while operating the healthcare monitoring system 10, because the router 700 may support two different communication protocols according to one embodiment of the present invention. The same approach applies not only to the locating method, but also to all embodiment of the present invention.

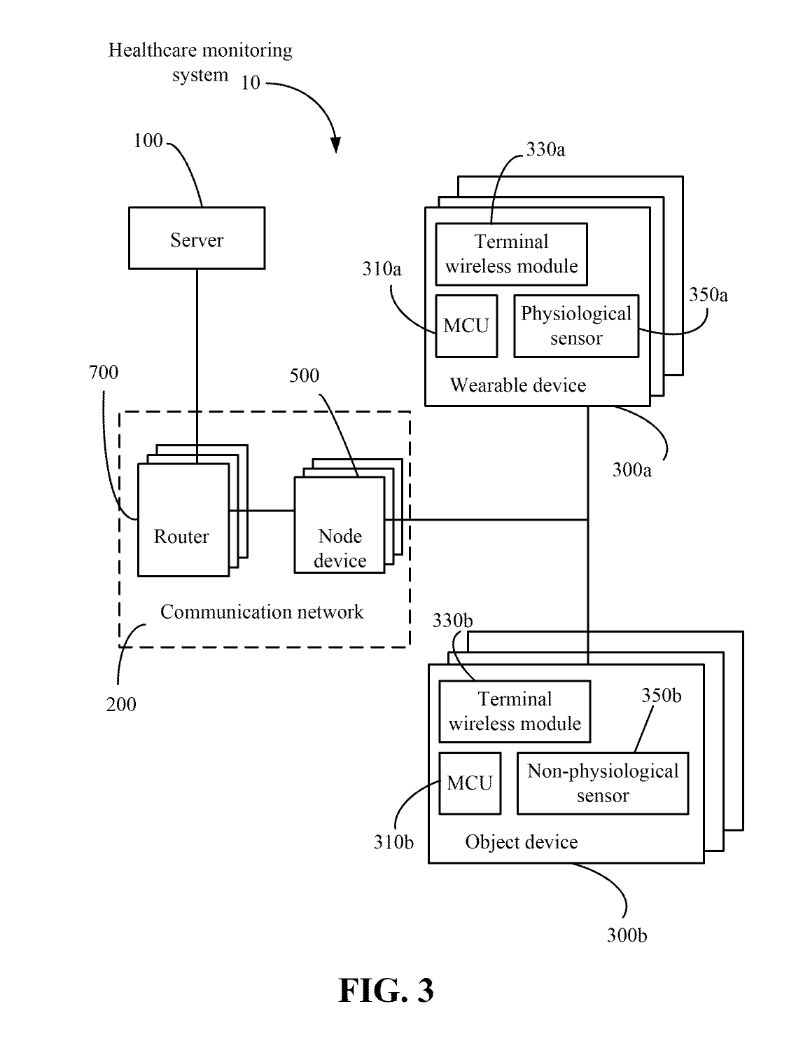

FIG. 3 schematically shows a healthcare monitoring system 10 according to one embodiment of the present invention, wherein the healthcare monitoring system 10 further comprises a wearable device 300a and an object device 300b instead of the terminal device 300 comparing to FIG. 1. The wearable device 300a may comprise a MCU 310a, a terminal wireless module 330a, and a physiological sensor 350a connected to one another. The object device 300b may comprise a MCU 310b, a terminal wireless module 330b, and a non-physiological sensor 350b connected to one another. The physiological sensor 350a may be sensor for body temperature, pulse, respiration rate, blood pressure, blood glucose, blood oxygen, etc. The non-physiological sensor 350b may be sensor for proximity, force, pressure, touch, optical, humidity, room temperature, oxygen, carbon dioxide, image, sound, photon, etc.

In one embodiment of the present invention, the physiological sensor 350a may sense a physiological signal, and pass the physiological signal to the MCU 310a. The MCU 310a may generate a physiological parameter based on the physiological signal. And the terminal wireless module 330a may send the physiological parameter to the node device 500. Then, the node device 500 may send the physiological parameter to the server 100. The physiological parameter may be saved in the storage 150 for monitoring record. At the same time, the physiological parameter may be checked against a preconfigured value by the processor 110. Alternatively, the physiological parameter may be checked against previous physiological parameter saved in the storage 150. If the processor 110 determines the physiological parameter as abnormal comparing to the preconfigured value or previous monitoring record in the storage 150, the server 110 may send a notification to the wearable device 300a. Therefore, the healthcare monitoring system 10 may provide monitoring of physiological parameter to a user of the wearable device 300a. In the case of remote healthcare monitoring, the server 100 may send the notification and the current location of the wearable device 300a to a healthcare provider such as doctor, nurse, or even the user's family to notify about the abnormal physiological parameter, wherein the current location of the wearable device 300a may be determined by the locating method as illustrated in FIG. 2. For example, the wearable device 300a may comprise a temperature sensor as the physiological sensor 350a, and may send a physiological parameter such as 38.5° C. to the server 100. The preconfigured value may be 37° C., so the server 100 determines the user's body temperature as abnormal. Thus, a notification may be sent to the wearable device 300a to notify the user. Alternatively, the notification and the current location may be sent to the healthcare provider by the server 100 to an external computing device, so the healthcare provider may be able to locate the user and provide healthcare assistance such as antipyretic in this case. Alternatively, the wearable device 300a may further comprise an actuator (not shown) such as a hypodermic syringe, and the hypodermic syringe may be actuated when the wearable device 300a detects an abnormal physiological parameter such as blood glucose level too high or too low. Therefore the hypodermic syringe may inject the user with insulin or sugar. The same approach may apply with different type of actuator based on user's medical condition, such as high blood pressure, high blood potassium, Systemic Lupus Erythematosus, etc. Especially for user needs high attention all the time such as people with disease which may cause serious health problem or even sudden death, the healthcare monitoring system 10 may provide real time monitoring of the user and corresponding emergency response.

In one embodiment of the present invention, the non-physiological sensor 350b may sense a non-physiological signal, and pass the non-physiological signal to the MCU 310b. The MCU 310b may generate non-physiological information based on the non-physiological signal. And the terminal wireless module 330b may send the non-physiological information to the node device 500. Then, the node device 500 may send the non-physiological information to the server 100. The non-physiological information may be saved in the storage 150 for monitoring record. The object device 300b may provide monitoring of environment such as room temperature with the non-physiological sensor 350b regarded as a temperature sensor, wherein any other type of the non-physiological sensors 350b may also be applied for different monitoring purposes. For example, the object device 300b may be installed on the back of a chair or cushion of a chair, wherein the object device 300b may comprise a pressure sensor as the non-physiological sensor 350b. When someone sits on the chair with one's back against the back of the chair, the pressure sensor sends a pressure signal to the MCU 310b. Thus, non-physiological information such as “chair occupied” may be generated by the MCU 310b according to the pressure signal. The object device 300b sends the non-physiological information to the server 100, so the server 100 knows someone is sitting on the chair.

In one embodiment of the present invention, the healthcare monitoring system 10 may provide monitoring of the user's behavior. This is achieved by applying the locating method in FIG. 2 for both the wearable device 300a and the object device 300b, wherein the user is wearing the wearable device 300a. For example, the object device 300b with a proximity sensor as the non-physiological sensor 350b may be installed at a bath tub (not shown). When the user approaches the bath tub, the server 100 may receive a non-physiological information as “someone at the bath tub” from the object device 300b. The locating method allows the server 100 to know the current location of the bath tub where the object device 300b is installed, and the current location of the user wearing the wearable device 300a. By matching the current location of both the object device 300b and wearable device 300a, the server 100 may confirm the user is at the bath tub. It should be noted that all monitoring of the healthcare monitoring system 10 is real time and dynamic. For example, the current location and non-physiological information may be continuously determined and sent to the server 100, so the server 100 may provide time of the user was approaching the bath tub, and time of the user leaving the bath tub. Thus, the duration of the user at the bath tub may be determined by the server 100. In the case of the healthcare monitoring system 10 comprises more than one wearable device 300a, the server 100 may even determine who is at the bath tub. This is achieved by recognizing the difference between each wearable device 300a using any identification information, wherein the identification information may be device ID such as serial number, MAC address of the terminal wireless module 330a, or identification of the user saved in the MCU 310a, etc. In view of the above, a habit information of the user may be generated by the server 100 based on the user's behavior over time. For example, the user's daily routine such as meal time, exercise time, shower time, bed time, etc may be determined by the server 100 as the habit information. Once the server 100 realizes the user is not following the habit information determined previously, a notification may be sent to the wearable device 300a of the user. Alternatively, the server 100 may be configured to remind the user about the daily routine by sending notification to the wearable device 300a, so the user may be able to keep the same habit. Furthermore, the server 100 may send a notification to the healthcare provider about changing of habit, so the healthcare provider may be able to respond.

In one embodiment of the present invention, the physiological parameter generated by the wearable device 300a may be further processed by the server 100 for generating health condition by the processor 110. The physiological parameter may be such as skin impedance, pulse, respiration, blood pressure, or biological mark, lactic acid level, etc, wherein health status of the user may be determined by the server 100 based on at least one of the physiological parameter or any combination of the physiological parameters. Thus, the health condition of the user may be generated by the server 100 by determining the user's health status over time. For example, the user's blood pressure, pulse, and heart rate are monitored every five minutes by the wearable device 300a, and the wearable device 300a sends the monitored result to the server 100 every 5 minutes to save in the storage 150. The server 100 determines the health status of the user based on the result, so the health status of the user over time would be generated by the server 100 to be the health condition of the user.

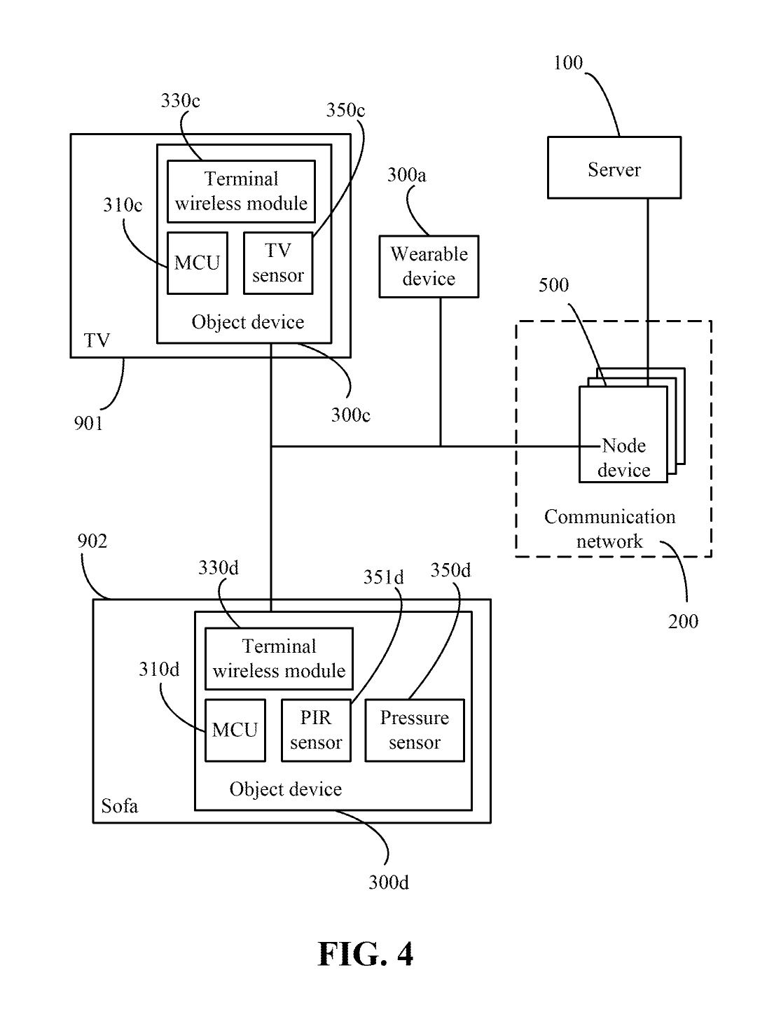

FIG. 4 schematically shows the healthcare monitoring system 10 that is installed with one object device 300c at a TV 901 and one object device 300d at a sofa 902 in front of the TV 901 according to one embodiment of the present invention. The health status and the behavior of the user may be further processed by the processor 110 of the server 100. The server 100 may combine the health status and the behavior to generate health information. Thus, the health information may indicate the relationship between the user's health status and behavior. For example, the object device 300c at the TV 901 may comprise a TV sensor 350c to sense whether the TV 901 is ON or OFF, and the object device 300d at the sofa 902 may comprise a pressure sensor 350d and a passive infrared (PIR) sensor 351d to sense if any living being is sitting on it. The TV sensor 350c may sense ON signal and send to a MCU 310c, so the object device 300c may send non-physiological information as “TV is ON” to the server 100. The pressure sensor 350d may sense a pressure signal and send to a MCU 310d, and the PIR sensor 351d may sense an infrared signal and send to the MCU 310d, so the object device 300d may send non-physiological information as “Sofa being pressed” and “Living being is around” respectively to the server 100. Therefore, the server 100 may determine “a living is sitting on the sofa 902 watching the TV 901” based on the above non-physiological information. When the user of the wearable device 300a is sitting on the sofa 902 watching the TV 901, the user's behavior may be sensed by the object device 300c and the object device 300d, and this is further confirmed by the current location of the wearable device 300a and the current location of the object device 300d, wherein the current location may be determined by the locating method in FIG. 2. At the same time, the physiological parameter such as heart rate may be sent by the wearable device 300a to the server 100, so the server 100 may further determine the health status of the user based on the heart rate, wherein the health status may be “normal heart rate”. Thus, the server 100 may combine the health status and the behavior to determine the health information, wherein the health information indicates “the user is watching the TV 901 with normal heart rate” in this case. In another case, if the heart rate sent by the wearable device 300a to the server 100 is determined as abnormal, then the server 100 may determine the health information as “the user is watching the TV 901 with abnormal heart rate”. Therefore, the server 100 may send a notification to a healthcare provider with the current location of the wearable device 300a to notify the healthcare provider to check out whether the user is having arythmia or not due to watching the TV 901.

In one embodiment of the present invention, the health condition and the habit information may be further processed by the processor 110 to determine a health monitoring record. As mentioned before, the health condition is the health status of the user over time, and the habit information is the behavior of the user over time, wherein the health status may combine with the behavior to become the health information. Therefore, health monitoring record is the health information over time. For example, the user with the wearable device 300a may monitor the user's heart rate with the physiological sensor 350a, and the object device 300b may monitor the user's bath time with the non-physiological sensor 350b. Furthermore, the object device 300b may further comprise a second non-physiological sensor (not shown) to monitor the user's bath temperature. Thus the user's bath time and the bath temperature with the heart rate while having the bath may be recorded by the server 100 and saved in the storage 150. One day, the user may experience heart attack at the bath tub with an abnormal heart rate lower than the heart rate in the health monitoring record determined previously by the server 100, at the same time the bath temperature sent to the server 100 is 10° C. that is lower than usual as well comparing to the health monitoring record. Thus, the server 100 may notify a healthcare provider to rescue the user. Then, the healthcare provider may be able to understand reason of the heart attack from the health monitoring record after being notified.

In one embodiment of the present invention, the physiological parameter from the wearable device 300a may be further processed, wherein the server 100 may determine an emotion indicator by the processor 110 based on the physiological parameter. For example, the server 100 may determine the emotion indicator according to the user's skin impedance, skin temperature, heart rate, respiration rate, etc.

In one embodiment of the present invention, the healthcare provider or the user's family may be able to access the server 100 for real-time monitoring of the user with the wearable device 300a, wherein the access may be granted by a set of account name and account password. The account name and account password may be preconfigured by the user and bound to the wearable device 300a. The access may be possible through internet, Ethernet, etc with an external computing device. The access to the server 100 may also provide the user's health monitoring record to the healthcare provider or user's family. Thus, when the health monitoring record shows abnormal health information over time, the healthcare provider or the user's family may make a medical appointment to a medical service provider that is connected to the healthcare monitoring system 10. Furthermore, a point-of-care device (not shown) may be connected to the healthcare monitoring system 10, wherein the user may use the point-of-care device under the healthcare provider's advice for treatment or regular health examination.

In one embodiment of the present invention, the health monitoring record provides detail reference for a doctor or the healthcare provider to understand what happened to the user. For example, the user has dehydrating symptom, and the doctor may check the health monitoring record to know if the user took enough water by counting how many times the user approached a water fountain with the object device 300b installed. The same approach may be applied by installing the object device 300b with a presence sensor at a toilet, so the server 100 may provide health monitoring record with how many times the user went to toilet. It should be noted that all user behavior may be recorded by the healthcare monitoring system 10 as long as the behavior related object is installed with the object device 300b with corresponding sensor. Furthermore, the doctor may combine the health monitoring system with external medical database such as medical history to understand about the user's health problem in a more detail manner.

In another embodiment of the present invention, the health monitoring record may also shows the user's medical compliance by comprising the object device 300b installed at medication. Thus, the health monitoring record may provide the time and even the dose of the medication that the user took. Changing of medical compliance may result in health problems, so the health monitoring record helps the healthcare provider to monitor and prevent the user to have health problem or even make existing health problem worse. Furthermore, the wearable device 300a may further comprise a tag reader (not shown) to read a NFC or RFID tag on the medication, wherein the NFC or RFID tag may provide corresponding information of the medication such as name of drug, property of drug, etc. Thus, the user may scan the tag to ensure correct medication to be taken. Alternatively, the wearable device 300a may comprise a medication concentration sensor (not shown) to monitor the actual amount of medication taken by the user. Also, a camera unit (not shown) may be included in the healthcare monitoring system 10 to monitor the user's medical compliance.

In view of the above, the health monitoring record may comprise water drinking frequency, an eating condition, a mood swing, a sleep condition, an exercise condition, and a defecation and urination condition, etc.

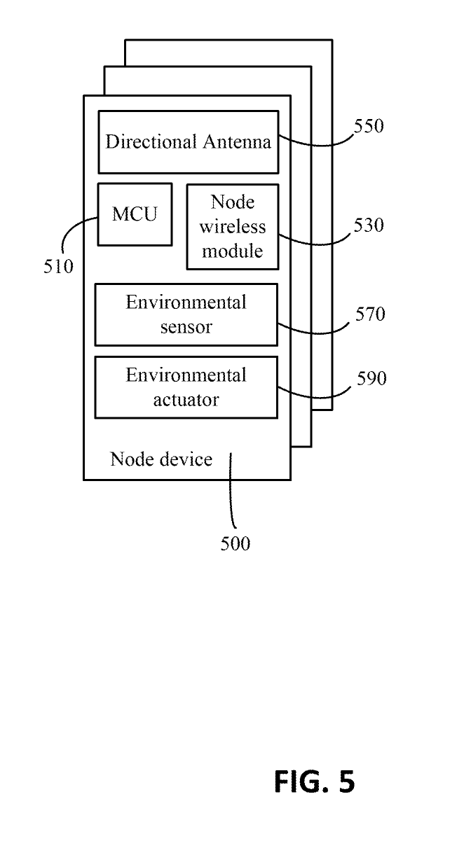

FIG. 5 schematically shows the node device 500 further comprising an environmental sensor 570 and an environmental actuator 590 according to one embodiment of the present invention. The environmental sensor 570 may be a humidity sensor, room temperature sensor, carbon dioxide sensor, camera, microphone, photometer, hygrometer, thermometer, etc. The environmental actuator 590 may be configured to control the environmental factor corresponding to the type of the environmental sensor 570, such as a heater, dehumidifier, ventilator, air conditioner, illumination device, stereo set, multimedia system, etc. For example, if the environmental sensor 570 is a temperature sensor, then the environmental actuator 590 may be a heating unit or a cooling unit or the combination of the two. The environmental sensor 570 may sense a temperature signal and send it to the MCU 510, wherein the MCU 510 may generate environmental information such as “room temperature 35° C.” based on the environmental signal. Then, the environmental information may be sent to the server 100 and saved in the storage 150 for monitoring purposes. Furthermore, the server 100 may comprise a preconfigured room temperature value according to user preference, so the environmental actuator 590 may control the room temperature according an environmental adjustment signal from the server 100, wherein the environmental adjustment signal is generated by the processor 110 according to the preconfigured room temperature value. Alternatively, the server 100 may generate the environmental adjustment signal based on the health information mentioned above. For example, the health information may indicate “user watching TV with abnormal body temperature”, and the server 100 may send the environmental adjustment signal to the environmental actuator 590 to higher or lower the room temperature for the user. In another case, the server 100 may receive the environmental information such as “room temperature 20° C.” from the node device 500, and together with a physiological parameter such as “body temperature 39° C.” from the wearable device 300a. Therefore the server 100 may determine health information of the user as “user having fever”. So the server 100 may not send the environmental adjustment signal but send a notification to the healthcare provider instead. The server 100 may also send a notification to the user according to the environmental information. For example, the server 100 may notify the user to wear more cloth when the temperature sensed is lower than a preconfigured value. In view of the above, the node device 500 may use the environmental actuator 590 to control the environmental condition according to the environmental adjustment signal.

In one embodiment of the present invention, the node device 500 may further comprise a camera unit (not shown), wherein the camera unit may take at least one image of the user when the user is nearby. And the wearable device 300a may further comprise a motion sensor (not shown) such as an accelerometer, a digital compass, or a gyroscope to monitor the motion of the user. When the motion sensor senses a motion signal of the user, the wearable device 300a may send the motion signal to the server 100 via the node device 500. Then, the server 100 determines if the motion of the user is “abnormal” by the processor 110. Furthermore, the server 100 may confirm the motion of the user by the at least one image from the node device 500. Thus, if the motion of the user is determined as “abnormal”, a notification may be sent to the healthcare provider by the server 100. Abnormal motion may be falling, tripping, etc of the user.

In another embodiment of the present invention, the user of the wearable device 300a may be able to have a video communication via the server 100 with the healthcare provider that access the server 100 with a video communication enabled computing device. To realize the video communication, the wearable device 300a may further comprise a display (not shown), a camera (not shown), and a speaker (not shown). Alternatively, the node device 500 with the camera unit mentioned above may further comprise a speaker, so the user of the wearable device 300a may use the node device 500 for video communication with the healthcare provider.

It should be noted that, all wearable device 300a mentioned above may be substituted with an implanted device (not shown) instead, wherein the implanted device may perform like the wearable device 300a in all embodiment of the present invention. And all user mentioned may be human or animal in all embodiment of the present invention. The animal may be pet, domestic animal, or raise animal, etc.

Previous descriptions are only embodiments of the present invention and are not intended to limit the scope of the present invention. Many variations and modifications according to the claims and specification of the disclosure are still within the scope of the claimed invention. In addition, each of the embodiments and claims does not have to achieve all the advantages or characteristics disclosed. Moreover, the abstract and the title only serve to facilitate searching patent documents and are not intended in any way to limit the scope of the claimed invention.

What is claimed is:

1. A healthcare monitoring system, comprising:
a server comprising:
a communication module;
a processor;
a storage connected to the processor and the communication module;
a node device comprising:
a first microcontroller unit (MCU);
a first wireless module;
a directional antenna connected to the first MCU and the first wireless module;
a terminal device, comprising:
a second MCU;
a second wireless module;
a physiological sensor connected to the wearable MCU and the wearable wireless module;
an object device, comprising:
a third MCU;
a third wireless module;
a non-physiological sensor connected to the third MCU and the third wireless module;
wherein when the physiological sensor senses a physiological signal, the second MCU generates a physiological parameter based on the physiological signal, and the second wireless module sends the physiological parameter to the node device,
wherein when the non-physiological sensor senses a non-physiological signal, the third MCU generates a non-physiological information based on the non-physiological signal, and the third wireless module sends the non-physiological information to the node device,
wherein the node device receives and sends the physiological parameter and the non-physiological information to the server by the first wireless module,
wherein the server determines a health information of a user by the processor based on the physiological parameter and the non-physiological information.

2. The healthcare monitoring system according to claim 1, wherein the physiological parameter and the non-physiological information are saved in the storage.

3. The healthcare monitoring system according to claim 1, wherein the server sends a notification to the terminal device by the communication module if the health information is abnormal.

4. The healthcare monitoring system according to claim 1, wherein the server sends a notification to a healthcare provider by the communication module if the health information is abnormal.

5. The healthcare monitoring system according to claim 1, wherein the node device broadcasts location information to the terminal device by the directional antenna, and the terminal device determines a current location by the second MCU according to the location information and a RSSI, and the terminal device sends the current location to the server by the second wireless module.

6. The healthcare monitoring system according to claim 5, wherein the server sends a notification and the current location to a healthcare provider by the communication module if the health information is abnormal.

7. The healthcare monitoring system according to claim 1, wherein the server further generates habit information based on the non-physiological information, and further generates health condition based on the physiological parameter, and wherein a health monitoring record is determined based on the habit information and the health condition.

8. The healthcare monitoring system according to claim 1, wherein the terminal device further comprises a motion sensor to detect motion of the user of the terminal device and to provide a motion signal to the server via the node device, and wherein when the motion is determined as abnormal, the server sends a notification to a healthcare provider.

9. The healthcare monitoring system according to claim 8, wherein the node device further comprises a camera unit to provide at least one image to the server to monitor the motion of the user, and wherein the motion is determined based on the motion signal and the at least one image.

10. The healthcare monitoring system according to claim 1, wherein the node device further comprises an environmental sensor that senses an environmental signal, the first MCU generates an environmental information based on environmental signal, and the first wireless module sends the environmental information to the server, and wherein the server determines the health information of the user based on the physiological parameter and the environmental information.

11. The healthcare monitoring system according to claim 10, wherein the node device further comprises an environmental actuator that controls environmental condition based on an environmental adjustment signal from the server, and wherein the environmental adjustment signal is determined based on the health information.

12. The healthcare monitoring system according to claim 1, wherein the terminal device is a wearable device.

13. The healthcare monitoring system according to claim 1, wherein the terminal device is an implanted device.

14. A method for healthcare monitoring, comprising:
receiving, by a server, a physiological parameter from a terminal device having a physiological sensor;
receiving, by the server, a non-physiological information from an object device having a non-physiological sensor;
determining, by the server, a health information of a user of the terminal device by a processor based the physiological parameter and the non-physiological information.

15. The method for healthcare monitoring according to claim 14, further comprising:
sending, by the server, a notification to the terminal device if the health information is abnormal.

16. The method for healthcare monitoring according to claim 14, further comprising:
sending, by the server, a notification to a healthcare provider if the health information is abnormal.

17. The method for healthcare monitoring according to claim 14, further comprising:
receiving, by the server, a current location that is determined by the terminal device;
sending, by the server, a notification and the current location to a healthcare provider if the health information is abnormal.

18. The method for healthcare monitoring according to claim 14, further comprising:
generating, by the server, habit information based on the non-physiological information;
generating, by the server, health condition based on the physiological parameter;
generating, by the server, a health monitoring record based on the habit information and health condition.

19. The method for healthcare monitoring according to claim 14, further comprising:
receiving, by the server, a motion signal from the terminal device having a motion sensor;
determining, by the server, a motion based on the motion signal;
determining, by the server, whether the motion is abnormal;
sending, by the server, a notification to a healthcare provider if the motion is determined as abnormal.

20. The method for healthcare monitoring according to claim 19, further comprising:
receiving, by the server, at least one image from a camera unit installed on a node device;
determining, by the server, whether the motion is abnormal based on the at least one image and the motion signal.

21. The method for healthcare monitoring according to claim 14, further comprising:
receiving, by the server, an environmental information from a node device having an environmental sensor;
determining, by the server, a health information of a user of the terminal device by a processor based on the physiological parameter and the environmental information.

22. The method for healthcare monitoring according to claim 21, further comprising:
determining, by the server, an environmental adjustment signal based on the health information;
sending, by the server, the environmental adjustment signal to the node device having an environmental actuator, wherein the node device controls environmental condition by the environmental actuator based on the environmental adjustment signal.

23. The method for healthcare monitoring according to claim 14, wherein the terminal device is a wearable device.

24. The method for healthcare monitoring according to claim 14, wherein the terminal device is an implanted device.

25. A node device for healthcare monitoring system, comprising:
a microcontroller unit (MCU);
a wireless module;
a directional antenna connected to the MCU and the wireless module,
wherein the directional antenna broadcasts location information to a terminal device, receives a current location from the terminal device, and sends the current location to a server,
wherein the current location is determined by the terminal device according to the location information.

26. The node device for healthcare monitoring system according to claim 25, further comprising an environmental sensor to sense an environmental signal, wherein the MCU generates an environmental information based on the environmental signal, and the wireless module sends the environmental information to the server.

27. The node device for healthcare monitoring system according to claim 26, further comprising an environmental actuator to control environmental condition, wherein the environmental actuator controls the environmental condition based on an environmental adjustment signal from the server.

28. The node device for healthcare monitoring system according to claim 25, further comprising a camera unit to monitor motion of a user of the terminal device, the camera unit provides at least one image to the server, and the server determines the motion based on the at least one image.

29. The node device for healthcare monitoring system according to claim 25, wherein the terminal device is a wearable device or an implanted device.

30. A terminal device for healthcare monitoring system, comprising:
a microcontroller unit (MCU);
a wireless module;
a physiological sensor connected to the MCU and the wireless module,
wherein, when the physiological sensor senses a physiological signal, the MCU generates a physiological parameter based on the physiological signal, and the wireless module sends the physiological parameter to a server via a node device, wherein the server determines health information of a user of the terminal device based on the physiological parameter, and sends a notification to the terminal device if the health information is abnormal.

31. The terminal device for healthcare monitoring system according to claim 30, wherein when the wireless module receives a location information from a node device, the MCU determines a current location based on the location information and RSSI of the location information, and the wireless module sends the current location to the server, and wherein the server sends a notification and the current location to a healthcare provider if the health information is abnormal.

32. The terminal device for healthcare monitoring system according to claim 30, further comprising a motion sensor to detect motion of the user and to provide a motion signal to the server via the node device, and wherein when the motion is determined as abnormal, the server sends a notification to a healthcare provider.

33. The terminal device for healthcare monitoring system according to claim 30, wherein the terminal device is a wearable device.

34. The terminal device for healthcare monitoring system according to claim 30, wherein the terminal device is an implanted device.
